(19) United States
(12) Patent Application Publication
(10) Pub. No.: US 2007/0265529 A1
(43) Pub. Date: Nov. 15, 2007

(54) ULTRASONIC DIAGNOSTIC APPARATUS AND IMAGE DISPLAY METHOD

(76) Inventor: Hiroshi Hashimoto, Tokyo (JP)

Correspondence Address: GENERAL ELECTRIC CO.;GLOBAL PATENT OPERATION, 187 Danbury Road, Suite 204, Wilton, CT, 06897-4122, US

(21) Appl. No.: 11/615,377

(22) Filed: Dec. 22, 2006

(30) Foreign Application Priority Data
May 11, 2006 (JP) 2006-133137

(51) Int. Cl. A61B 8/00

(52) U.S. Cl. 600/443

(57) ABSTRACT

To improve efficiency in diagnosis, a cross-sectional image of a subject is sequentially produced based on echo signals acquired by conducting a scan involving transmitting ultrasound to the subject and receiving ultrasound reflected from the subject. A region of interest is then defined in the cross-sectional image of the subject, and a TIC image representing an average brightness values of all pixels in the region of interest in the sequentially produced cross-sectional image of the subject is produced such that the TIC image corresponds to a time axis along which the echo signals are acquired. Moreover, a property-of-ultrasound image representing the property of the ultrasound sequentially transmitted to the region of interest is produced such that the property-of-ultrasound image corresponds to a time axis of the TIC image. The produced TIC image and property-of-ultrasound image are displayed on a screen next to each other such that their respective time axes correspond to each other.

### BACKGROUND OF THE INVENTION

The present invention relates to an ultrasonic diagnostic apparatus and an image display method.

An ultrasonic diagnostic apparatus is known as an apparatus for displaying a cross-sectional image or the like of a subject based on echo signals by ultrasound reflected from the subject to which ultrasound is transmitted. Since the ultrasonic diagnostic apparatus is capable of readily displaying a cross-sectional image in real time, it is widely used especially for medical applications such as checkup of a fetus or heart.

For the purpose of enhancing contrast of a region to be diagnosed to obtain a clearer and sharper image in examination using the ultrasonic diagnostic apparatus, imaging may be conducted with an ultrasonic contrast agent injected into a subject (such imaging will be sometimes referred to as contrast examination hereinbelow) to display the transition of the concentration of the contrast agent as a TIC (time intensity curve) image for diagnosis.

Exemplary ultrasonic contrast agents include microbubbles, for example. Microbubbles burst and disappear by receiving ultrasound of high intensity, i.e., ultrasound with a high sound pressure, and at the same time generate ultrasonic echoes of high intensity. In ultrasonic diagnosis by circulating an ultrasonic contrast agent within the subject, the procedure is sometimes carried out by combining an ultrasound transmission mode in which a behavior of the ultrasonic contrast agent is continuously observed without bursting the ultrasonic contrast agent or with a reduced factor of breakage, with another ultrasonic transmission mode in which appearance of the inside of the body is observed with the ultrasonic contrast agent broken or with an increased factor of breakage. In other words, diagnostic images are sometimes acquired by ultrasound transmitted into the subject with a varying property of ultrasound during a contrast examination (see Japanese Patent Application Laid Open No. 2002-306477, for example).

In such diagnosis by diagnostic images acquired with a varying property of ultrasound, the diagnosis is carried out by comparing a diagnostic image with an instant value of the property of ultrasound displayed on a screen display section displaying the diagnostic image.

A TIC image is a chart plotting the brightness value along a vertical axis and the time along a horizontal axis. A higher concentration of the contrast agent in a region to be diagnosed gives a higher brightness value represented along the vertical axis in the chart. Moreover, a varying property of ultrasound transmitted to the subject brings about a change of the brightness value represented along the vertical axis of the chart.

Therefore, if a contrast examination is performed with transmitted ultrasound of varying intensity, it is not easy to decide from the screen display section displaying the diagnostic image and instant value of the property of ultrasound whether the change of the brightness value results from a change of the concentration of the contrast agent in a region to be diagnosed in the subject or it results from a change of the property of ultrasound transmitted to the subject, leading to reduced efficiency in diagnosis.

### SUMMARY OF THE INVENTION

It is therefore an object of the present invention to provide an ultrasonic diagnostic apparatus and an image display method capable of improving efficiency in diagnosis in producing a TIC image for diagnosis even with a varying property of ultrasound transmitted to a subject.

To attain the aforementioned object, an ultrasonic diagnostic apparatus according to the present invention comprises: a scan section for sequentially acquiring echo signals by conducting a scan involving sequentially transmitting ultrasound to a subject and sequentially receiving ultrasound reflected from said subject to which said ultrasound is sequentially transmitted; a cross-sectional image producing section for sequentially producing a cross-sectional image of said subject based on said echo signals acquired by said scan section; a region-of-interest defining section for defining a region of interest in said cross-sectional image of said subject sequentially produced by said cross-sectional image producing section; a TIC image producing section for producing a TIC image representing brightness values in the region of interest defined by said region-of-interest defining section in said cross-sectional image sequentially produced by said cross-sectional image producing section such that said TIC image corresponds to a time axis along which said echo signals are acquired by said scan section; a property-of-ultrasound image producing section for producing a property-of-ultrasound image representing a property of said ultrasound sequentially transmitted to said region of interest by said scan section such that said property-of-ultrasound image corresponds to a time axis of said TIC image produced by said TIC image producing section; and an image display section for displaying said TIC image produced by said TIC image producing section and property-of-ultrasound image produced by said property-of-ultrasound image producing section next to each other on a screen such that their respective time axes correspond to each other.

To attain the aforementioned object, an image display method according to the present invention comprises: a first step of sequentially producing a cross-sectional image of a subject based on echo signals acquired by conducting a scan involving sequentially transmitting ultrasound to said subject and sequentially receiving ultrasound reflected from said subject to which said ultrasound is sequentially transmitted; a second step of defining a region of interest in said cross-sectional image of said subject sequentially produced at said first step; a third step of producing a TIC image representing brightness values in said region of interest defined at said second step in said cross-sectional image of said subject sequentially produced at said first step such that said TIC image corresponds to a time axis along which said echo signals are acquired; a fourth step of producing a property-of-ultrasound image representing a property of said ultrasound sequentially transmitted to said region of interest such that said property-of-ultrasound image corresponds to a time axis of said TIC image produced at said third step; and a fifth step of displaying said TIC image produced at said third step and said property-of-ultrasound image produced at said fourth step next to each other on a screen such that their respective time axes correspond to each other.

According to the present invention, there is provided an ultrasonic diagnostic apparatus and an image display method capable of improving efficiency in diagnosis in producing a TIC image for diagnosis even with a varying property of ultrasound transmitted to a subject.

Further objects and advantages of the present invention will be apparent from the following description of the preferred embodiments of the invention as illustrated in the accompanying drawings.

### BRIEF DESCRIPTION OF THE DRAWINGS

FIG. 1 is a block diagram showing the configuration of an ultrasonic diagnostic apparatus of an embodiment in accordance with the present invention.

FIG. 2 is a flow chart of an image display method in an embodiment in accordance with the present invention.

FIG. 3 shows an exemplary display screen displayed by the image display section 40 in a first embodiment in accordance with the present invention.

FIG. 4 shows an exemplary display screen displayed by the image display section 40 in a second embodiment in accordance with the present invention.

FIG. 5 shows an exemplary display screen displayed by the image display section 40 in a third embodiment in accordance with the present invention.

FIG. 6 shows an exemplary display screen displayed by the image display section 40 in a fourth embodiment in accordance with the present invention.

### DETAILED DESCRIPTION OF THE INVENTION

Now several embodiments in accordance with the present invention will be described with reference to the accompanying drawings.

### First Embodiment

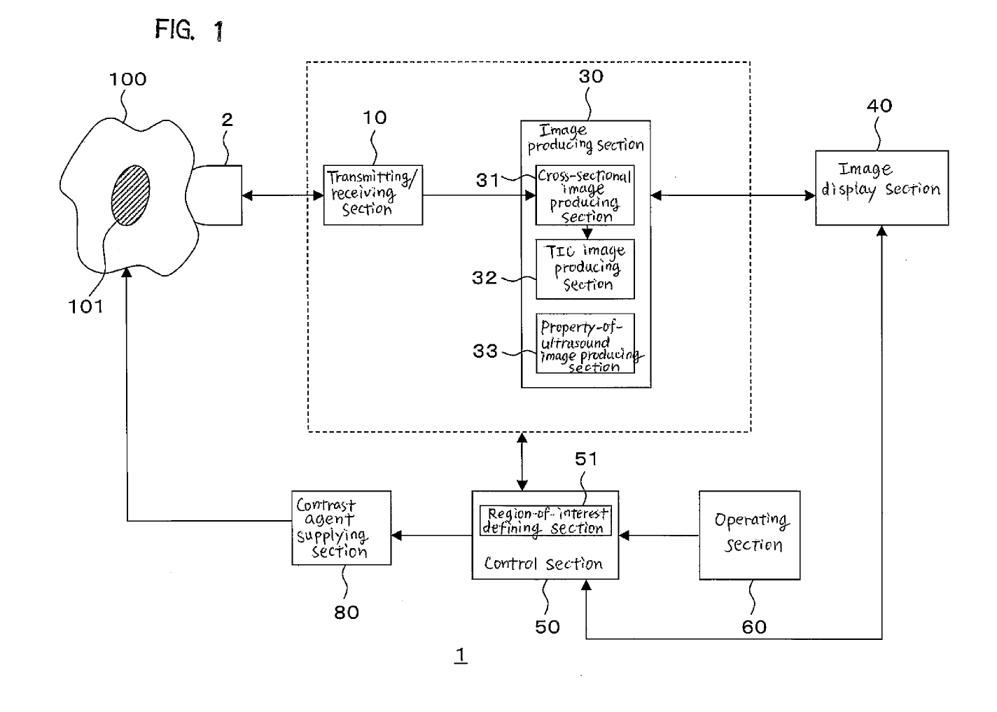

FIG. 1 is a block diagram showing the configuration of an embodiment of an ultrasonic diagnostic apparatus in accordance with the present invention.

As shown in FIG. 1, an ultrasonic diagnostic apparatus 1 comprises an ultrasonic probe 2, a transmitting/receiving section 10, an image producing section 30, an image display section 40, a control section 50, an operating section 60, and a contrast agent supplying section 80.

These components will now be described one by one hereinbelow.

The ultrasonic probe 2 is made by arranging a plurality of ultrasonic transducers uniformly in a matrix. The ultrasonic transducers formed in the ultrasonic probe 2 are made of a piezoelectric material such as a PZT (lead zirconate titanate) ceramic, for example. The ultrasonic probe 2 is used by putting its face having the ultrasonic transducers against the surface of the subject 100.

The ultrasonic probe 2 transmits ultrasound from the ultrasonic transducers to the subject 100 by a drive signal sent from the transmitting/receiving section 10 in response to a command supplied by the control section 50. Then, ultrasound reflected by the subject 100 to which the ultrasound is transmitted is received and converted into echo signals by the ultrasonic transducers. The ultrasonic probe 2 then transmits the echo signals to the transmitting/receiving section 10.

The transmitting/receiving section 10 is connected to the ultrasonic probe 2. The transmitting/receiving section 10 supplies a drive signal to the ultrasonic probe 2 to transmit ultrasound, in response to a command supplied by the control section 50. The transmitting/receiving section 10 also receives echo signals from the ultrasonic probe 2. It then applies processing such as amplification, delay, and addition to the received echo signals, and outputs them to the image producing section 30.

The image producing section 30 is connected to the transmitting/receiving section 10. The image producing section 30 is comprised of a computer and a program, for example. The image producing section 30 comprises a cross-sectional image producing section 31 for producing a cross-sectional image of a subject, a TIC image producing section 32 for producing a TIC image, and a property-of-ultrasound image producing section 33 for producing a property-of-ultrasound image, as shown in FIG. 1. The property-of-ultrasound image will be discussed in detail later.

The cross-sectional image producing section 31 sequentially receives echo signals from the transmitting/receiving section 10 in response to a command supplied by the control section 50. It applies logarithm amplification to the received echo signals, then detects their envelope, and sequentially converts them into echo intensity signals indicating echo intensity. It then uses the amplitude of the sequentially converted echo intensity signals as a brightness value and incorporates positional information into them to sequentially produce a cross-sectional image of the subject 100.

The TIC image producing section 32 determines an average brightness value of all pixels in a region of interest defined by a region-of-interest defining section 51 in the cross-sectional image sequentially produced by the cross-sectional image producing section 31. The average brightness values are arranged in a time series to produce a TIC image such that it corresponds to a time axis.

The property-of-ultrasound image producing section 33 applies image processing to property-of-ultrasound data input to the operating section 60 in response to a command supplied by the control section 50 to produce a property-of-ultrasound image.

The image display section 40 is connected to the image producing section 30. The image display section 40 is supplied with image signals from the image producing section 30, and displays an image based thereon. The image display section 40 comprises a CRT, a liquid crystal display or the like, which is capable of displaying a color image.

The image display section 40 displays the cross-sectional image sequentially produced by the cross-sectional image producing section 31, TIC image produced by the TIC image producing section 32, and property-of-ultrasound image produced by the property-of-ultrasound image producing section 33.

The image display section 40 simultaneously displays the TIC image and property-of-ultrasound image next to each other on the screen such that their respective time axes correspond to each other.

It should be noted that the cross-sectional image may be displayed simultaneously with the TIC image and property-of-ultrasound image, or it may be superimposed over them.

The control section 50 is comprised of a computer and a program, for example, and is connected to the aforementioned sections. The control section 50 supplies control signals to the sections in response to an operational signal supplied by the operating section 60 to control their operations. The control section 50 has a region-of-interest defining section 51, as shown in FIG. 1.

The region-of-interest defining section 51 defines a region of interest in a cross-sectional image produced by the cross-sectional image producing section 31. The region-of-interest defining section 51 defines the region of interest based on data representing a certain range of a cross-sectional image defined by an operation by the operator via the operating section 60, for example.

The operating section 60 is connected to the control section 50. The operating section 60 is comprised of several input devices such as, for example, a keyboard, a touch panel, a trackball, a foot switch, a vocal input device, etc. The operating section 60 is supplied with operational information from the operator, and outputs a command to the control section 50 based on the information.

For the purpose of enhancing contrast of a region to be diagnosed in the subject to obtain a clearer and sharper image, the contrast agent 101 may be supplied from the contrast agent supplying section 80 to the region to be diagnosed in the subject 100 for imaging.

The contrast agent supplying section 80 supplies the contrast agent 101 to the region to be diagnosed in the subject 100 based on a contrast agent supply signal output by the control section 50 in response to a command to supply the contrast agent 101 to the subject 100 input to the operating section 60 by the operator.

The contrast agent 101 is used for enhancing contrast of the region to be diagnosed in the diagnostic image. The contrast agent 101 has acoustic impedance significantly different from that of surrounding tissue in the subject 100, and is capable of selectively enhance contrast by supplying it to the subject 100. The contrast agent 101 comprises microbubbles, for example.

Now the operation of the ultrasonic apparatus of an embodiment in which diagnosis is conducted while supplying a contrast agent in accordance with the present invention will be discussed hereinbelow.

First, a scan procedure in the embodiment in which diagnosis is conducted while supplying a contrast agent in accordance with the present invention will be described.

The operator places the ultrasonic probe 2 at a predetermined position in the subject 100. The ultrasonic probe 2 is supplied with a drive signal from the transmitting/receiving section 10 in response to a command supplied by the control section 50 according to property-of-ultrasound data input to the operating section 60 by the operator. The ultrasonic probe 2 converts the drive signal supplied by the transmitting/receiving section 10 into ultrasound at ultrasonic transducers disposed in the ultrasonic probe 2, and sequentially transmits ultrasound to the subject 100.

While ultrasound is being sequentially transmitted to the subject 100, the contrast agent 101 is supplied from the contrast agent supplying section 80 to the subject 100 in response to a command supplied by the control section 50. At that time, a signal for supplying the contrast agent 101 to the subject 100 input by the operating section 60 to the control section 50 serves as a trigger to start measurement of an elapsed time.

Then, the ultrasound sequentially transmitted to the subject 100 is reflected at the subject 100, and an ultrasonic echo is received by the ultrasonic transducers in the ultrasonic probe 2. The received ultrasonic echo is converted into an echo signal at the ultrasonic transducers. The converted echo signal is output from the ultrasonic probe 2 to the transmitting/receiving section 10.

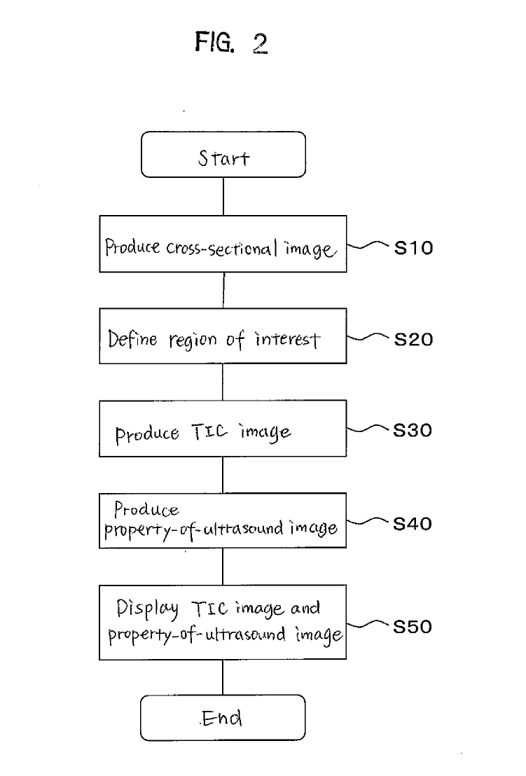

Now an image display method in an embodiment in accordance with the present invention will be described. FIG. 2 is a flow chart of the image display method in an embodiment in accordance with the present invention.

First, a cross-sectional image is produced (S10).

The transmitting/receiving section 10 outputs echo signals to the cross-sectional image producing section 31. The cross-sectional image producing section 31 then applies logarithm amplification to the echo signals, then detects their envelope, and sequentially converts them into echo intensity signals. The cross-sectional image producing section 31 then applies image processing to the converted echo intensity signals to sequentially produce a cross-sectional image.

A region of interest is next defined in the cross-sectional image (S20).

The region-of-interest defining section 51 defines a region of interest in the cross-sectional image produced by the cross-sectional image producing section 31. The region-of-interest defining section 51 defines the region of interest based on data representing a certain range in the cross-sectional image specified by a trackball, for example.

A TIC image for the region of interest defined in the cross-sectional image is next produced (S30).

The TIC image producing section 32 determines an average brightness value of all pixels in the region of interest defined in the cross-sectional image. The average brightness value data are then arranged in a time series, and the TIC image producing section 32 produces a TIC image such that the image corresponds to a time axis along which ultrasonic echoes are acquired by the ultrasonic probe 2. The time axis is established in the TIC image using a signal at which the contrast agent 101 is supplied to the subject 100 as a trigger.

Next, a property-of-ultrasound image is produced (S40).

The property-of-ultrasound data input to the operating section 60 by the operator is input to the property-of-ultrasound image producing section 33 in response to a command supplied by the control section 50. The property-of-ultrasound image producing section 33 applies image processing to the input property-of-ultrasound data. Thus, property-of-ultrasound image signals are obtained and a property-of-ultrasound image is produced such that the image corresponds to the time axis of the TIC image. The time axis is established in the property-of-ultrasound image using a signal at which the contrast agent 101 is supplied to the subject 100 as a trigger, which signal is input from the operating section 60 to the control section 50.

It should be noted that Step S40 is not necessarily conducted after Step S30, and it may be conducted in parallel with Step S10 through Step S30.

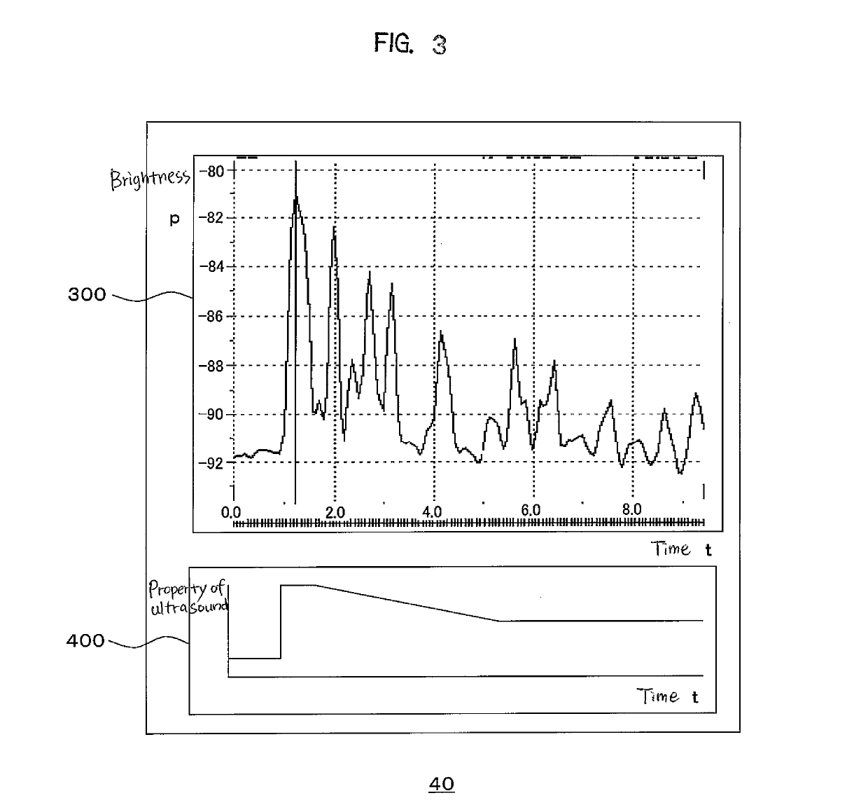

Next, as shown in FIG. 3, the TIC image 300 and property-of-ultrasound image 400 are displayed on the image display section 40 such that their respective time axes correspond to each other (S50).

The image display section 40 displays the TIC image 300 produced at Step S30 and property-of-ultrasound image 400 produced at Step S40 such that their respective time axes correspond to each other.

FIG. 3 is a diagram showing a display screen displayed by the image display section 40 in the first embodiment.

As shown in FIG. 3, the image display section 40 simultaneously displays the TIC image 300 and property-of-ultrasound image 400 in a top-bottom configuration. In this case, the property of ultrasound is an indication of intensity of ultrasound, such as for example, a mechanical index value (sometimes referred to as MI value hereinbelow), an acoustic output or a sound pressure.

The MI value is a mechanical indication representing a value normalized a maximum peak negative sound pressure on an acoustic axis by a referential sound pressure 1 MPa. It is also used as a value indicating an effect of ultrasound on a biological medium.

The MI value is generally defined as EQ. (1) below:
MI=P_{N}/(f_{c})^{1/2}, (1)
where P_{N} designates a peak value of a negative sound pressure (Pa), and f_{c} designates a frequency (Hz).

The acoustic output is energy of sound emanating from a sound source per unit time, and the energy of sound per unit area represents intensity of sound, which is proportional to a squire of the sound pressure. Therefore, the acoustic output is defined as EQ. (2) below:
P=α(P_{N})², (2)
where P designates an acoustic output, P_{N} designates a peak value of a negative sound pressure (Pa), and α is a factor of proportionality.

The TIC image 300 displayed on the image display section 40 in the first embodiment is a chart representing a temporal change of brightness, in which the vertical axis represents the brightness value p, and the horizontal axis represents the time t. The property-of-ultrasound image 400 is a line chart representing a temporal change of a property value of ultrasound, in which the vertical axis represents the property value of ultrasound, and the horizontal axis represents the time t. The TIC image 300 and property-of-ultrasound image 400 are simultaneously displayed on the image display section 40 such that their respective time axes correspond to each other.

In the first embodiment, as described above, echo signals are rendered as a cross-sectional image, and a region of interest is defined in the cross-sectional image. Then, a TIC image 300 of the defined region of interest is produced. A property value of ultrasound transmitted to the subject 100 is then represented in a line chart to produce a property-of-ultrasound image 400.

By representing the property-of-ultrasound image 400 as a line chart, intensity of ultrasound being transmitted to the subject can be easily recognized. Accordingly, by simultaneously displaying the TIC image 300 and property-of-ultrasound image 400 on the image display section 40 such that their respective time axes correspond to each other, it is easy to decide whether a change of brightness in the TIC image 300 results from a change of the concentration of the contrast agent in a region to be diagnosed in the subject 100, or it results from a change of the intensity of ultrasound transmitted by the ultrasonic probe 2. Thus, efficiency in diagnosis can be improved.

### Second Embodiment

A second embodiment of the ultrasonic diagnostic apparatus in accordance with the present invention will now be described in detail.

The present embodiment is similar to the first embodiment except the property-of-ultrasound image 400 displayed by the image display section 40. Accordingly, explanation of duplicated portions will be omitted.

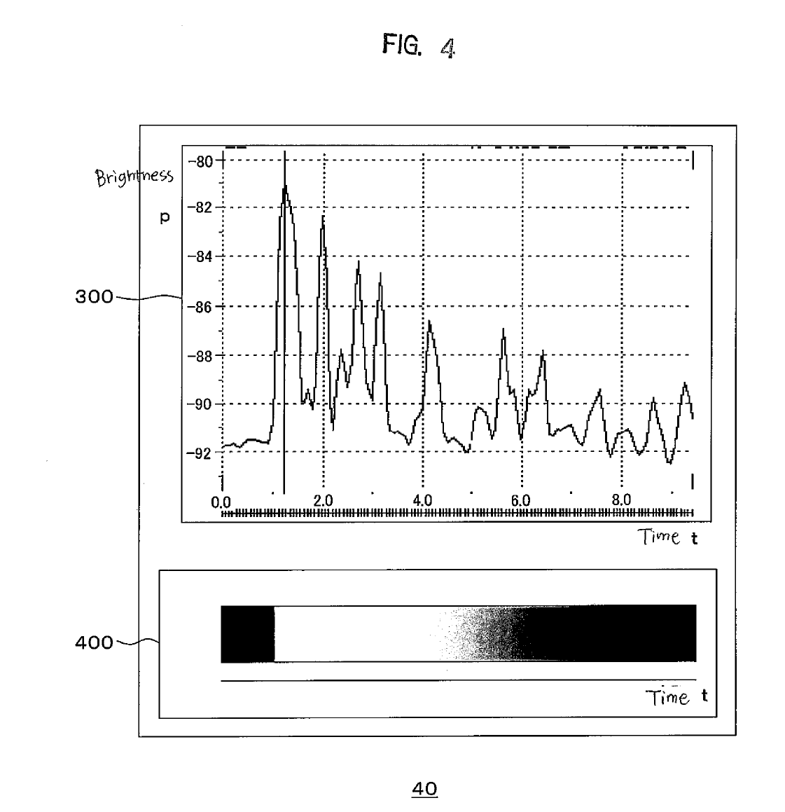

FIG. 4 is a diagram showing a display screen displayed by the image display section 40 in the second embodiment.

A property-of-ultrasound image 400 displayed on the image display section 40 in the second embodiment is a chart representing a temporal change of a property value of ultrasound, in which the horizontal axis represents the time t, and the temporal change is represented in a bar chart with a color concentration varying with the property value of ultrasound. The bar chart with a color concentration varying with the property value of ultrasound refers to a chart representing the property value of ultrasound using shading or tone or the like of color. It is a chart having, for example, a portion with a lighter color representing a higher property value of ultrasound, and a portion with a darker color representing a lower property value of ultrasound.

Again, the TIC image 300 and property-of-ultrasound image 400 are simultaneously displayed on the image display section 40 such that their respective time axes correspond to each other.

In the second embodiment, as described above, echo signals are rendered as a cross-sectional image, and a region of interest is defined in the cross-sectional image. Then, a TIC image 300 of the defined region of interest is produced. A property value of ultrasound transmitted to the subject 100 is then represented in a bar chart to produce a property-of-ultrasound image 400.

By representing the property-of-ultrasound image 400 as a bar chart, intensity of ultrasound being transmitted to the subject can be easily recognized, as in the first embodiment. Accordingly, by simultaneously displaying the TIC image 300 and property-of-ultrasound image 400 on the image display section 40 such that their respective time axes correspond to each other, it is easy to decide whether a change of brightness in the TIC image 300 results from a change of the concentration of the contrast agent in a region to be diagnosed in the subject 100, or it results from a change of the intensity of ultrasound transmitted by the ultrasonic probe 2. Thus, efficiency in diagnosis can be improved.

### Third Embodiment

A third embodiment of the ultrasonic diagnostic apparatus in accordance with the present invention will now be described in detail.

The present embodiment is similar to the first embodiment except the property-of-ultrasound image 400 displayed by the image display section 40. Accordingly, explanation of duplicated portions will be omitted.

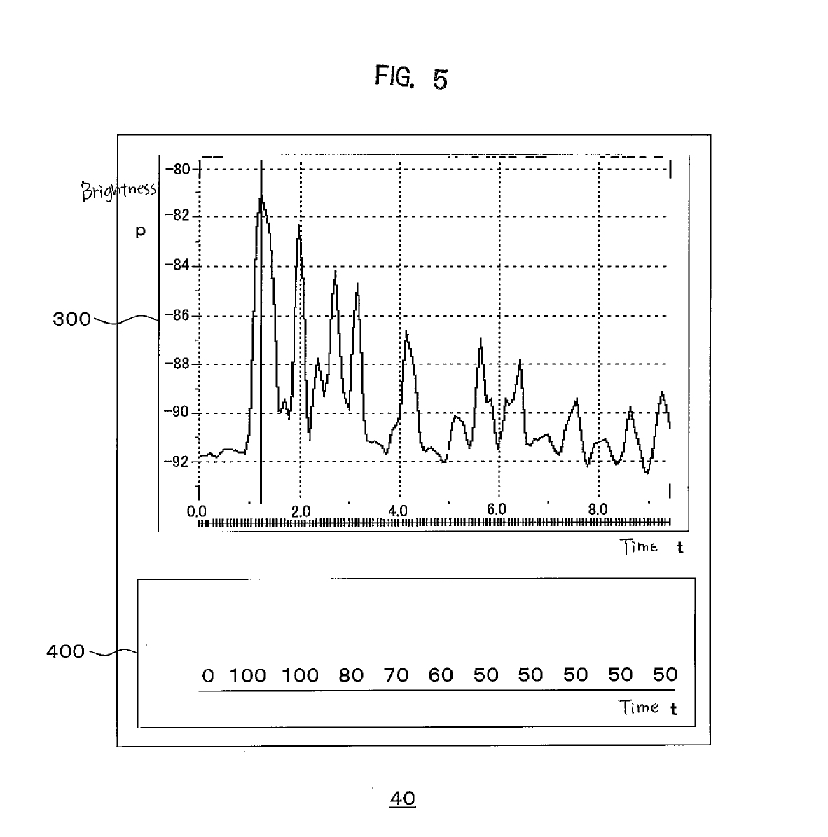

FIG. 5 is a diagram showing a display screen displayed by the image display section 40 in the third embodiment.

A property-of-ultrasound image 400 displayed on the image display section 40 in the third embodiment comprises numeric values representing a temporal change of a property value of ultrasound, in which the horizontal axis represents the time t, and the temporal change is represented as numeric values indicating the property value of ultrasound at regular intervals. The representation by numeric values indicating the property value of ultrasound at regular intervals refers to the property value of ultrasound acquired at regular intervals, and the property value of ultrasound is represented on the image display section 40 as numeric values.

Again, the TIC image 300 and property-of-ultrasound image 400 are simultaneously displayed on the image display section 40 such that their respective time axes correspond to each other.

In the third embodiment, as described above, echo signals are rendered as a cross-sectional image, and a region of interest is defined in the cross-sectional image. Then, a TIC image 300 of the defined region of interest is produced. A property of ultrasound transmitted to the subject 100 is then represented as numeric values indicating a property value of ultrasound at regular intervals to produce a property-of-ultrasound image 400.

By representing the property-of-ultrasound image 400 as numeric values indicating the property value of ultrasound at regular intervals, intensity of ultrasound being transmitted to the subject can be easily recognized, as in the first embodiment. Accordingly, by simultaneously displaying the TIC image 300 and property-of-ultrasound image 400 on the image display section 40 such that their respective time axes correspond to each other, it is easy to decide whether a change of brightness in the TIC image 300 results from a change of the concentration of the contrast agent in a region to be diagnosed in the subject 100, or it results from a change of the intensity of ultrasound transmitted by the ultrasonic probe 2. Thus, efficiency in diagnosis can be improved.

### Fourth Embodiment

A fourth embodiment of the ultrasonic diagnostic apparatus in accordance with the present invention will now be described in detail.

The present embodiment is similar to the first embodiment except the property-of-ultrasound image 400 displayed by the image display section 40. Accordingly, explanation of duplicated portions will be omitted.

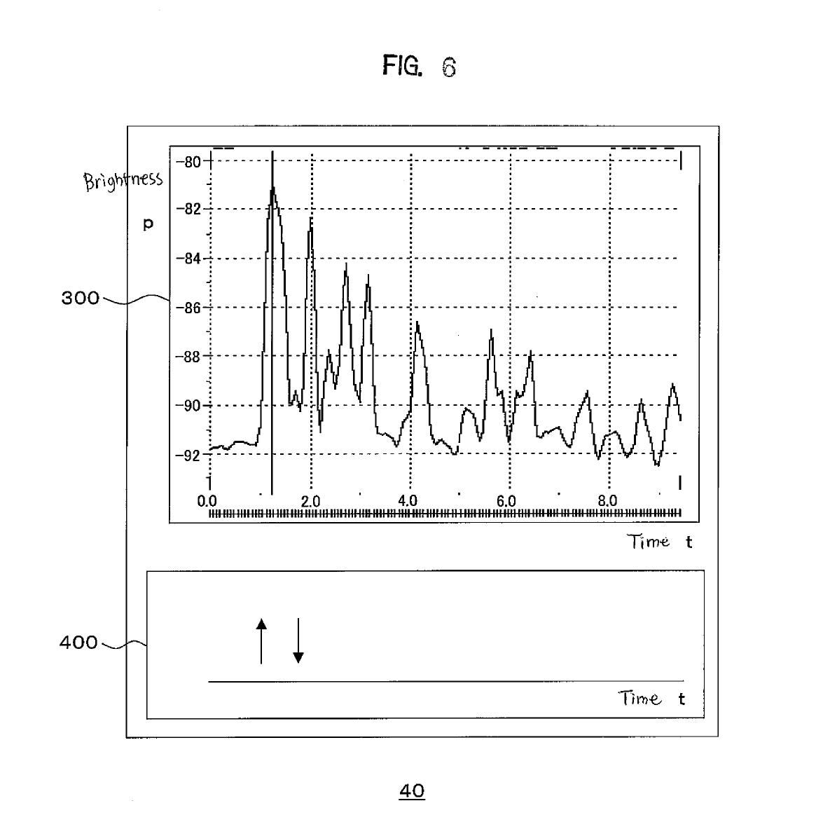

FIG. 6 is a diagram showing a display screen displayed by the image display section 40 in the fourth embodiment.

A property-of-ultrasound image 400 displayed on the image display section 40 in the fourth embodiment is a chart in which the horizontal axis represents the time t, and a start point of transition of a property value of ultrasound is represented. The start point of transition of a property value of ultrasound refers to a chart representing the time at which the property value of ultrasound starts to change such that the time corresponds to the time axis of the TIC image 300. Note that the start point of transition represented by an up-pointing arrow indicates that the property value of ultrasound increases, and that represented by a down-pointing arrow indicates that the property value of ultrasound decreases.

Again, the TIC image 300 and property-of-ultrasound image 400 are simultaneously displayed on the image display section 40 such that their respective time axes correspond to each other.

In the fourth embodiment, as described above, echo signals are rendered as a cross-sectional image, and a region of interest is defined in the cross-sectional image. Then, a TIC image 300 of the defined region of interest is produced. A property of ultrasound transmitted to the subject 100 is then represented as the start point of transition of the property value of ultrasound to produce a property-of-ultrasound image 400.

By representing the property-of-ultrasound image 400 as the start point of transition of the property value of ultrasound, transition of intensity of ultrasound transmitted to the subject can be easily recognized. Accordingly, by simultaneously displaying the TIC image 300 and property-of-ultrasound image 400 on the image display section 40 such that their respective time axes correspond to each other, it is easy to decide whether a change of the brightness value represented along the vertical axis of the chart in the TIC image 300 results from a change of the concentration of the contrast agent in a region to be diagnosed in the subject 10, or it results from a change of the intensity of ultrasound transmitted by the ultrasonic probe 2. Thus, efficiency in diagnosis can be improved.

It should be noted that the ultrasonic diagnostic apparatus 1 in the aforementioned embodiments corresponds to the ultrasonic diagnostic apparatus of the present invention. The ultrasonic probe 2 and transmitting/receiving section 10 in the aforementioned embodiments correspond to the scan section of the present invention. The cross-sectional image producing section 31 in the aforementioned embodiments corresponds to the cross-sectional image producing section of the present invention. The TIC image producing section 32 in the aforementioned embodiments corresponds to the TIC image producing section of the present invention. The property-of-ultrasound image producing section 33 in the aforementioned embodiments corresponds to the property-of-ultrasound image producing section of the present invention. The image display section 40 in the aforementioned embodiments corresponds to the image display section of the present invention. Finally, the region-of-interest defining section 51 in the aforementioned embodiments corresponds to the region-of-interest defining section of the present invention.

The present invention is not limited to being practiced according to the aforementioned embodiments, and several variations may be employed.

Although the step of supplying the contrast agent 101 to the subject 100 is employed in the embodiments of the present invention, the step is conducted for the purpose of enhancing contrast of the region to be diagnosed to obtain a clearer and sharper image, and therefore, the object of the present invention may be attained without conducting the step. Accordingly, the step may be omitted. In this case, a trigger to define a time axis for producing an image may be a signal input to the operating section 60 by the operator for transmitting ultrasound to the subject.

Moreover, although the property of ultrasound is exemplified by the MI value, acoustic output and sound pressure in the embodiments of the present invention, the present invention is not limited thereto and any other appropriate indication that allows recognition of intensity of ultrasound may be used.

Furthermore, although, in the embodiments of the present invention, the property-of-ultrasound image is, for example, a line chart in the first embodiment, the present invention is not limited thereto and any other appropriate image that allows recognition of the property of ultrasound, such as, for example, a plot of a temporal change of the property value of ultrasound, may be used.

Many widely different embodiments of the invention may be configured without departing from the spirit and the scope of the present invention. It should be understood that the present invention is not limited to the specific embodiments described in the specification, except as defined in the appended claims.

1. An ultrasonic diagnostic apparatus comprising:
a scan section for sequentially acquiring echo signals by conducting a scan involving sequentially transmitting ultrasound to a subject and sequentially receiving ultrasound reflected from said subject to which said ultrasound is sequentially transmitted;
a cross-sectional image producing section for sequentially producing a cross-sectional image of said subject based on said echo signals acquired by said scan section;
a region-of-interest defining section for defining a region of interest in said cross-sectional image of said subject sequentially produced by said cross-sectional image producing section;
a TIC image producing section for producing a TIC image representing brightness values in the region of interest defined by said region-of-interest defining section in said cross-sectional image sequentially produced by said cross-sectional image producing section such that said TIC image corresponds to a time axis along which said echo signals are acquired by said scan section;
a property-of-ultrasound image producing section for producing a property-of-ultrasound image representing a property of said ultrasound sequentially transmitted to said region of interest by said scan section such that said property-of-ultrasound image corresponds to a time axis of said TIC image produced by said TIC image producing section; and
an image display section for displaying said TIC image produced by said TIC image producing section and property-of-ultrasound image produced by said property-of-ultrasound image producing section next to each other on a screen such that their respective time axes correspond to each other.

2. The ultrasonic diagnostic apparatus of claim 1, wherein:
the property of said ultrasound is a mechanical index value.

3. The ultrasonic diagnostic apparatus of claim 1, wherein:
the property of said ultrasound is an acoustic output.

4. The ultrasonic diagnostic apparatus of claim 1, wherein:
the property of said ultrasound is a sound pressure.

5. The ultrasonic diagnostic apparatus of claim 1, wherein:
said property-of-ultrasound image is a line chart.

6. The ultrasonic diagnostic apparatus of claim 1, wherein:
said property-of-ultrasound image is a bar chart with a color concentration varying with a property value of ultrasound.

7. The ultrasonic diagnostic apparatus of claim 1, wherein:
said property-of-ultrasound image comprises numeric values indicating said property value of ultrasound at regular intervals.

8. The ultrasonic diagnostic apparatus of claim 1, wherein:
said property-of-ultrasound image comprises a start point of transition of said property value of ultrasound.

9. An image display method, comprising:
a first step of sequentially producing a cross-sectional image of a subject based on echo signals acquired by conducting a scan involving sequentially transmitting ultrasound to said subject and sequentially receiving ultrasound reflected from said subject to which said ultrasound is sequentially transmitted;
a second step of defining a region of interest in said cross-sectional image of said subject sequentially produced at said first step;
a third step of producing a TIC image representing brightness values in said region of interest defined at said second step in said cross-sectional image of said subject sequentially produced at said first step such that said TIC image corresponds to a time axis along which said echo signals are acquired;
a fourth step of producing a property-of-ultrasound image representing a property of said ultrasound sequentially transmitted to said region of interest such that said property-of-ultrasound image corresponds to a time axis of said TIC image produced at said third step; and
a fifth step of displaying said TIC image produced at said third step and said property-of-ultrasound image produced at said fourth step next to each other on a screen such that their respective time axes correspond to each other.

10. The image display method of claim 9, wherein:
the property of said ultrasound is a mechanical index value.

11. The image display method of claim 9, wherein:
the property of said ultrasound is an acoustic output.

12. The image display method of claim 9, wherein:
the property of said ultrasound is a sound pressure.

13. The image display method of claim 9, wherein:
said property-of-ultrasound image is a line chart.

14. The image display method of claim 9, wherein:
said property-of-ultrasound image is a bar chart with a color concentration varying with said property value of ultrasound.

15. The image display method of claim 9, wherein:
said property-of-ultrasound image comprises numeric values indicating said property value of ultrasound at regular intervals.

16. The image display method of claim 9, wherein:
said property-of-ultrasound image represents a start point of transition of said property value of ultrasound.
